(19) United States
(12) Patent Application Publication
(10) Pub. No.: US 2025/0082278 A1
(43) Pub. Date: Mar. 13, 2025

(54) EQUANIMITY - DYNAMIC PHYSIOLOGY

(71) Applicant: Arizona Board of Regents on Behalf of the University of Arizona, Tucson, GA (US)

(72) Inventors: John Allen, Tucson, AZ (US); Richard Lane, Tucson, AZ (US); Janet Roveda, Tucson, AZ (US); Ping Chang, Tucson, AZ (US); Shu-Fen Wung, Tucson, AZ (US)

(21) Appl. No.: 18/824,616

(22) Filed: Sep. 4, 2024

Related U.S. Application Data
(60) Provisional application No. 63/581,182, filed on Sep. 7, 2023.

(51) Int. Cl. A61B 5/00; A61B 5/024

(52) U.S. Cl. CPC A61B 5/7267; A61B 5/02405; A61B 5/7203

(57) ABSTRACT

Disclosed is a heart rate monitoring system containing a machine learning platform containing self-learning and/or personalizable algorithms capable of detecting and filtering out artifacts in signals indicative of an individual's heart rate, such signals generated from one or more electrocardiogram sensors. The self-learning and/or personalizable algorithms are updated periodically. Accordingly, the machine learning platform improves its accuracy in detecting and/or rejecting/filtering out artifacts and/or becomes more personalized as further signals indicative of an individual's heart rate are processed. The machine learning platform performs detection and filtering out of artifacts via dimension reduction algorithms. The system further contains a first algorithm for real time measurement of heart rate variability (HRV). Also disclosed is a heart rate monitoring method that implements a heart rate monitoring system disclosed herein. The heart rate monitoring system and method can partition physiological components of HRV from other non-physiological components, particularly physical activity and/or breathing.

### CROSS-REFERENCE TO RELATED APPLICATION

This application claims benefit of and priority to U.S. Provisional Application No. 63/581,182 filed Sep. 7, 2023, the contents of which is incorporated herein by reference in its entirety.

### FIELD OF THE INVENTION

This invention is in the field of heart rate monitoring systems; particularly heart rate monitoring systems containing self-learning and/or personalizable algorithms for improved measurements of an individual's heart rate variability.

### BACKGROUND OF THE INVENTION

Modern electronics are ubiquitous in healthcare. For example, monitoring devices often include electronic components and algorithms to sense, measure, and monitor living beings. Monitoring equipment can measure vital signs such as respiration rate, oxygen level in the blood, heart rate, and so on. Not only are monitoring devices used in the clinical setting, monitoring devices are also used often in sports equipment and consumer electronics.

One important measurement performed by many of the monitoring equipment is heart rate, typically measured in beats per minute (BPM). Athletes use heart rate monitors to get immediate feedback on a workout, while health care professionals use heart rate monitors to monitor the health of a patient. Many solutions for measuring heart rate are available on the market today. For instance, electronic heart rate monitors can be found in the form of chest straps and watches. However, these electronic heart rate monitors are often not very accurate, due to a high amount of noise present in the signals provided by the sensors of these monitors. The noise is often caused by the fact that the user is moving and also by the lack of secure contact between the monitor and the user. This noisy environment often leads to an irregular, inaccurate or even missing readout of the heart rate.

Therefore, it is an object of the invention to provide an improved heart monitoring system that can analyze data and make health-based interventions to an individual in need thereof.

It is also an object of the invention to provide an improved heart monitoring system that can analyze data and provide personalized health-based interventions to an individual in need thereof.

It is another object of the invention to provide an improved heart monitoring system containing a self-learning and/or personalizable algorithms to provide personalized health-based interventions to an individual in need thereof.

Therefore, it is an object of the invention to provide an improved heart monitoring method that can analyze data and make health-based interventions to an individual in need thereof.

It is also an object of the invention to provide an improved heart monitoring method that can analyze data and provide personalized health-based interventions to an individual in need thereof.

It is another object of the invention to provide an improved heart monitoring method containing a self-learning and/or personalizable algorithms to provide personalized health-based interventions to an individual in need thereof.

### SUMMARY OF THE INVENTION

Disclosed is a heart rate monitoring system containing a machine learning platform containing self-learning and/or personalizable algorithms capable of detecting and/or rejecting/filtering out artifacts in signals indicative of an individual's heart rate, such signals generated from one or more electrical sensors, such as electrocardiogram sensors. The heart rate monitoring system contains one or more sensors operably linked to a portable device that is in turn operably linked to the machine learning platform located in a desktop computer, a laptop computer, or a cloud computing server. Accordingly, the device is configured to transmit data to the machine learning platform. The device is also configured to receive data from the one or more sensors, the machine learning platform, or a combination thereof. Therefore, the device effectively receives data (such as one or more signals indicative of an individual's heartbeat) from one or more sensors, transmits the data to the machine learning platform, and gets data from the machine learning platform. To receive and/or transmit these data, one or more apps (e.g., a custom-built app) are installed in the device for reception and transmission of the data from the sensors and to the machine learning platform, respectively. The data from the machine learning platform can be heart rate variability measurements, physical interventions, cognitive interventions, or a combination thereof. These data are preferably transmitted to the device, when the one or more signals indicative of heart rate indicate that heart rate variability measure falls below a threshold indicative of a cardiac disorder.

Preferably, the artifacts are interference with the one or more sensors, mechanical interactions with the one or more sensors, premature heartbeats, bumping of a body part against an object, or a combination thereof.

In the non-limiting example involving stress management, a diffusion model can be utilized to eliminate potential noises in the sensor measurements. The diffusion model is a better fit compared to other noise elimination models as it is based on the concept that a continuous Gaussian diffusion process can be reversed with the same functional form as the forward process. By modeling the reverse process, pure noise will converge to data points sampled from the observed data distribution. Given a large enough number of diffusion steps, this can be represented with a discrete Gaussian diffusion process. In the instant disclosure, a Transformer-based Diffusion Probabilistic Model can be used, that implements a deep learning technique known as the Transformer. During the reverse process, noise resources are tracked, tagged, and the noise patterns identified. The proposed diffusion approach models the physiological data as time series and divides it into forward and reverse trajectories. Conditioned on the history observation (e.g., already measured data), the proposed method learns the noise-adding process as a Markov chain and reverse the trajectory to get true and clean measurements.

The self-learning and/or personalizable algorithms are updated periodically. Accordingly, the machine learning platform improves its accuracy in detecting and/or rejecting/filtering out artifacts and/or becomes more personalized as further signals indicative of an individual's heart rate are processed. The machine learning platform performs detection and/or rejection/filtering out of artifacts via dimension reduction algorithms, such as a Markov model.

The heart rate monitoring system contains a first algorithm operably linked to the machine learning platform. The first algorithm is capable of generating one or more heart rate variability measurements from the one or more signals in real time, such as within 1, 2, 3, 4, 5, 10, 15, 20, or no more than 30 minutes after receiving one or more signals indicative of an individual's heart rate.

Also disclosed is a heart rate monitoring method that implements a heart rate monitoring system disclosed herein. Preferably, the heart rate monitoring method is capable of partitioning physiological components of heart rate variability from other non-physiological components, such as physical activity and/or breathing.

### DETAILED DESCRIPTION OF THE INVENTION

### I. Definitions

“Heart rate monitoring system” refers to a system that provides heart rate measurements (such as heart rate variability measurements) of a subject. An example of a heart monitoring system is one that provides these measurements in real time, such as within 1, 2, 3, 4, 5, 10, 15, 20, or no more than 30 minutes after receiving one or more signals indicative of heart rate.

“Operably linked” refers to the connection of at least two components in the disclosed heart rate monitoring system via technology including, but not limited to, internet, ethernet, Bluetooth, near field communication, WiFi, integrated circuits, or a combination thereof.

“Personalizable,” as relates to an algorithm, refers to an algorithm that can be customized to detect stimuli involving an individual's circumstances (e.g., physical activities) as the algorithm encounters more data regarding the individual's circumstances (e.g., physical activities).

### II. Heart Rate Monitoring System or Method

Disclosed is a heart rate monitoring system containing a machine learning platform capable of detecting and/or rejecting/filtering out artifacts in one or more signals indicative of heart rate, wherein the one or more signals are generated by one or more sensors operably linked to the machine learning platform. Advantageously, the machine learning platform contains self-learning and/or personalizable algorithms capable of detecting and/or rejecting/filtering out the artifacts. The artifacts include, but are not limited to, interference with the one or more sensors, mechanical interactions with the one or more sensors, premature heartbeats, bumping of a body part against an object, or a combination thereof. Preferably, the artifacts are interference with the one or more sensors, mechanical interactions with the one or more sensors, premature heartbeats, bumping of a body part against an object, or a combination thereof.

In general, artifacts such as potential noises from one or more sensors measurements disclosed herein, can be rejected/filtered out/eliminated by utilizing a diffusion model. A diffusion model can be distinguished from generative models and variational inference as follows. Generative models aim to learn the underlying distribution of an observation dataset, but the main challenge lies in calculating the normalizing constant, which is intractable. Variational inference is a commonly used solution, transforming distribution calculation into an optimization problem. It starts with a randomly parameterized distribution and adjusts its parameters during training until it approaches the true distribution. In the current application, the diffusion model is a better fit as it is based on the concept that a continuous Gaussian diffusion process can be reversed with the same functional form as the forward process. By modeling the reverse process, pure noise will converge to data points sampled from the observed data distribution. Given a large enough number of diffusion steps, this can be approximated with a discrete Gaussian diffusion process. In the instant disclosure, a Transformer-based Diffusion Probabilistic Model can be used, that implements a deep learning technique known as the Transformer. During the reverse process, noise resources are tracked, tagged, and the noise patterns identified. The proposed diffusion approach models can use physiological data as time series and divide it into forward and reverse trajectories. Conditioned on the history observation (e.g., already measured data), the proposed method learns the noise-adding process as a Markov chain and reverses the trajectory to get true and clean measurements.

The self-learning and/or personalizable algorithms can also be updated as needed or periodically. Accordingly, the machine learning platform improves its accuracy in detecting and/or rejecting/filtering out artifacts and/or becomes more personalized as further signals indicative of an individual's heart rate are processed. The machine learning platform performs detection and/or rejection/filtering out of artifacts preferably via dimension reduction algorithms. Some dimension reduction algorithms include, but are not limited to, a Markov model, Scikit-Learn Library Installation, Classification Dataset, Principal Component Analysis, Singular Value Decomposition, Linear Discriminant Analysis, Isomap Embedding, Locally Linear Embedding, Modified Locally Linear Embedding, or a combination thereof. Preferably, the dimension reduction algorithm includes a Markov model. The machine learning platform can train its model(s) in a supervised, semi-supervised, or unsupervised manner. In some forms, the machine learning platform contains neural networks selected from recurrent neural networks, convolutional neural networks, and artificial neural networks.

In some forms, the heart rate monitoring system contains a first algorithm operably linked to the machine learning platform. The first algorithm is capable of generating one or more heart rate variability measurements from the one or more signals in real time, such as within 1 min, 2 mins, 3 mins, 4 mins, 5 mins, 10 mins, 15 mins, 20 mins, or no more than 30 mins after receiving one or more signals indicative of an individual's heart rate. Heart rate variability is an index of vagal control of the heart that is a marker for physical and mental health. Measures of vagal control of the heart are affected by physical activity as well as psychological stress and current measures of heart rate variability do not attempt to disentangle them in a self-learning and/or personalized way that optionally includes breathing and/or respiration. As a result, current measures of heart rate variability provide a weak marker of psychological stress. Further, the first algorithm is capable of detecting and/or partitioning the one or more heart rate variability measurements that are due to physical activity, respiration, breathing, or a combination thereof. In preferred forms, the first algorithm specifically detects and/or partitions the one or more heart rate variability measurements that are due to respiration, breathing, or a combination thereof.

Preferably, the machine learning platform is in a desktop computer, a laptop computer, or a cloud computing server.

In some forms, of the heart rate monitoring system, the one or more sensors include, but are not limited to, electrical sensors (e.g., electrocardiogram (ECG)) optical sensors, audio sensors, capacitive sensors, magnetic sensors, chemical sensors, humidity sensors, moisture sensors, pressure sensors, and/or biosensors. Preferably, the one or more sensors include electrical sensors (e.g., ECG).

In some forms, the heart rate monitoring system contains a device, such as a portable device, e.g., mobile phone operably linked to the one or more sensors and/or the machine learning platform. Preferably, the device is operably linked to the one or more sensors and the machine learning platform. Accordingly, the device can be configured to transmit data to the machine learning platform. The device can also be configured to receive data from the one or more sensors, the machine learning platform, or a combination thereof. Therefore, the device can effectively receive data (such as one or more signals indicative of an individual's heartbeat) from one or more sensors, transmit the data to the machine learning platform, and get data from the machine learning platform. To receive and/or transmit these data, one or more apps (e.g., a custom-built app) can be installed in the device for reception and transmission of the data from the sensors and to the machine learning platform, respectively.

The device can also receive data from the machine learning platform. Preferably, one or more apps installed on the device provide this capability. The data from the machine learning platform can be heart rate variability measurements, physical interventions, cognitive interventions, or a combination thereof. These data are preferably transmitted to the device, when the one or more signals indicative of heart rate indicate that heart rate variability measure falls below a threshold indicative of a cardiac disorder. The threshold can be 30th percentile for an individual's age group. “Age group” can be ages that fall within 10 years, 7.5 years, 5 years, 2.5 years, or 1 year of the individual's age.

The heart rate monitoring system or parts thereof can take many different forms. Examples include watches, rings, wristbands, chest straps, headbands, headphones, ear buds, clamps, clips, clothing, bags, shoes, glasses, googles, hats, suits, necklace, attachments/patches/strips/pads which can adhere to a living being, accessories, portable devices, and so on. In particular, wearables technology (or referred often as “wearables”, i.e., electronics which are intended to be worn by humans or other living beings) can greatly leverage the benefits of the heart rate monitoring device disclosed herein due to the wearables' portability and the heart rate monitoring technique's robustness against motion artifacts and/or heart rate variability component arising from breathing and/or respiration. Even in the presence of noise, the heart rate monitoring system can effectively track a heart rate. Besides wearables, portable or mobile devices such as mobile phones and tablets can also include a processor having the tracking functions, an analog front end, a light source and a light sensor (or an extension (wired or wireless) having the light source and light sensor) to provide a heart rate monitoring system or a part thereof. Furthermore, it is envisioned that the heart rate monitoring system can be used in wired or wireless accessories such as cuffs, clips, straps, bands, probes, etc., to sense physiological parameters of a living being.

Also disclosed is a heart rate monitoring method that implements a heart rate monitoring system disclosed herein. Preferably, the heart rate monitoring method is capable of partitioning physiological components of heart rate variability from other non-physiological components, such as physical activity and/or breathing.

The disclosed heart rate monitoring system or method can be further understood through the following enumerated paragraphs or embodiments.
1. A heart rate monitoring system containing a machine learning platform capable of detecting and/or rejecting/filtering out artifacts in one or more signals indicative of heart rate that are received by the heart rate monitoring system and processed by the machine learning platform, wherein the one or more signals are generated by one or more sensors operably linked to the machine learning platform.
2. The heart rate monitoring system of paragraph 1, wherein the machine learning platform contains a diffusion model, such as a diffusion probabilistic model.
3. The heart rate monitoring system of paragraph 1 or 2, wherein the detection and/or rejection/filtering out of the artifacts involves dimension reduction, preferably involving a Markov model.
4. The heart rate monitoring system of any one of paragraphs 1 to 3, wherein the machine learning platform contains self-learning and/or personalizable algorithms for detection and/or rejection/filtering out of the artifacts.
5. The heart rate monitoring system of any one of paragraphs 1 to 4, wherein the machine learning platform improves its accuracy in detecting and/or rejecting/filtering out artifacts and/or becomes more personalized as further signals indicative of heart rate processed by the machine learning platform.
6. The heart rate monitoring system of any one of paragraphs 1 to 5, further containing a first algorithm capable of generating one or more heart rate variability measurements from the one or more signals in real time, such as within 1, 2, 3, 4, 5, 10, 15, 20, or no more than 30 minutes after the one or more signals are received by the heart rate monitoring system, wherein the first algorithm is operably linked to the machine learning platform.
7. The heart rate monitoring system of paragraph 6, wherein the first algorithm is capable of detecting and/or partitioning one or more heart rate variability measurements that are due to physical activity, respiration, breathing, or a combination thereof from the one or more heart rate variability measurements.
8. The heart rate monitoring system of paragraph 6 or 7, wherein the first algorithm is capable of detecting and/or partitioning one or more heart rate variability measurements that are due to respiration, breathing, or a combination thereof from the one or more heart rate variability measurements.
9. The heart rate monitoring system of any one of paragraphs 1 to 8, wherein the machine learning platform is in a desktop computer, a laptop computer, or a cloud computing server.
10. The heart rate monitoring system of any one of paragraphs 1 to 9, wherein the one or more sensors contain electrical sensors (e.g., electrocardiogram (ECG)), optical sensors, audio sensors, capacitive sensors, magnetic sensors, chemical sensors, humidity sensors, moisture sensors, pressure sensors, and/or biosensors, preferably electrical sensors (e.g., ECG).
11. The heart rate monitoring system of any one of paragraphs 1 to 10, further containing a device, such as a hand-held (e.g., mobile phone), operably linked to the one or more sensors and/or the machine learning platform.
12. The heart rate monitoring system of paragraph 11, wherein the device is configured to receive data from the one or more sensors, the machine learning platform, or a combination thereof.
13. The heart rate monitoring system of paragraph 11 or 12, wherein the device is configured to transmit data to the machine learning platform.
14. The heart rate monitoring system of any one of paragraphs 1 to 13, wherein the artifacts contain interference with the one or more sensors, mechanical interactions with the one or more sensors, premature heartbeats, bumping of a body part against an object, or a combination thereof.
15. The heart rate monitoring system of any one of paragraphs 1 to 14, capable of providing physical and/or cognitive interventions when the heart rate variability measure falls below a threshold, such as the 30th percentile for an individual's age group.
16. The heart rate monitoring system of paragraph 15, wherein the physical and/or cognitive interventions involve meditation recommendations, relaxation recommendations, breathing recommendations, reappraisal of a situation, or a combination thereof.
17. A heart rate monitoring method comprising the heart rate monitoring system of any one of paragraphs 1 to 16, the method comprising: partitioning physiological components of heart rate variability from other non-physiological components, such as physical activity and/or breathing.

We claim:

1. A heart rate monitoring system comprising a machine learning platform capable of detecting and/or rejecting/filtering out artifacts in one or more signals indicative of heart rate that are received by the heart rate monitoring system and processed by the machine learning platform, wherein the one or more signals are generated by one or more sensors operably linked to the machine learning platform.

2. The heart rate monitoring system of claim 1, wherein the machine learning platform comprises a diffusion model, such as a diffusion probabilistic model.

3. The heart rate monitoring system of claim 1, wherein the detection and/or rejection/filtering out of the artifacts comprises dimension reduction, preferably involving a Markov model.

4. The heart rate monitoring system of claim 1, wherein the machine learning platform comprises self-learning and/or personalizable algorithms for detection and/or rejection/filtering out of the artifacts.

5. The heart rate monitoring system of claim 1, wherein the machine learning platform improves its accuracy in detecting and/or rejecting/filtering out artifacts and/or becomes more personalized as further signals indicative of heart rate processed by the machine learning platform.

6. The heart rate monitoring system of claim 1, further comprising a first algorithm capable of generating one or more heart rate variability measurements from the one or more signals in real time, such as within 1, 2, 3, 4, 5, 10, 15, 20, or no more than 30 minutes after the one or more signals are received by the heart rate monitoring system, wherein the first algorithm is operably linked to the machine learning platform.

7. The heart rate monitoring system of claim 6, wherein the first algorithm is capable of detecting and/or partitioning one or more heart rate variability measurements that are due to physical activity, respiration, breathing, or a combination thereof from the one or more heart rate variability measurements.

8. The heart rate monitoring system of claim 6, wherein the first algorithm is capable of detecting and/or partitioning one or more heart rate variability measurements that are due to respiration, breathing, or a combination thereof from the one or more heart rate variability measurements.

9. The heart rate monitoring system of claim 1, wherein the machine learning platform is in a desktop computer, a laptop computer, or a cloud computing server.

10. The heart rate monitoring system of claim 1, wherein the one or more sensors comprise electrical sensors (e.g., electrocardiogram (ECG)), optical sensors, audio sensors, capacitive sensors, magnetic sensors, chemical sensors, humidity sensors, moisture sensors, pressure sensors, and/or biosensors, preferably electrical sensors (e.g., ECG).

11. The heart rate monitoring system of claim 1, further comprising a device, such as a hand-held (e.g., mobile phone), operably linked to the one or more sensors and/or the machine learning platform.

12. The heart rate monitoring system of claim 11, wherein the device is configured to receive data from the one or more sensors, the machine learning platform, or a combination thereof.

13. The heart rate monitoring system of claim 11, wherein the device is configured to transmit data to the machine learning platform.

14. The heart rate monitoring system of claim 1, wherein the artifacts comprise interference with the one or more sensors, mechanical interactions with the one or more sensors, premature heartbeats, bumping of a body part against an object, or a combination thereof.

15. The heart rate monitoring system of claim 1, capable of providing physical and/or cognitive interventions when the heart rate variability measure falls below a threshold, such as the 30th percentile for an individual's age group.

16. The heart rate monitoring system of claim 15, wherein the physical and/or cognitive interventions comprise meditation recommendations, relaxation recommendations, breathing recommendations, reappraisal of a situation, or a combination thereof.

17. A heart rate monitoring method comprising the heart rate monitoring system of claim 1, the method comprising:
partitioning physiological components of heart rate variability from other non-physiological components, such as physical activity and/or breathing.
